(19) United States
(12) Patent Application Publication
(10) Pub. No.: US 2001/0022183 A1
(43) Pub. Date: Sep. 20, 2001

(54) Quick immobilization device

(76) Inventor: Luis Alberto Maya, Buenos Aires (AR)

Correspondence Address: Gregory P. LaPointe, BACHMAN & LaPOINTE, P.C., 900 Chapel Street, Suite 1201, New Haven, CT, 06510-2802, US

(21) Appl. No.: 09/812,739

(22) Filed: Mar. 20, 2001

(30) Foreign Application Priority Data
Mar. 20, 2000 (AR) P 00 0101231

(51) Int. Cl. A61F 13/00

(52) U.S. Cl. 128/846

(57) ABSTRACT

Quick immobilization device for immobilizing victims or patients to be moved or rescued, comprised by two main symmetrical parts and accessories, having an anatomic shape protecting the cranial, dorsum-lumbar regions and legs, which affords safety and avoids new lesions or worsening of the existing ones.

### FIELD OF THE INVENTION

The instant invention relates to a quick immobilization device used for protecting victims or patients who require immobilization to be moved or rescued, thus avoiding new lesions, progression of existing lesions or deterioration thereof.

### BACKGROUND OF THE INVENTION—PRIOR ART

Several immobilization devices are already known, all designed for affording safety in the immobilization and movement of accident victims or patients. The disadvantage in devices of prior art is that stabilization is carried out in several steps, thus increasing the time involved and the possibility of impairing existing lesions.

Existing devices comprise rigid tables with fixing straps. Once the patient is immobilized, cranial stabilizers, cervical collars (Philadelphia type), monitoring wiring, thermal blankets should be added to the device. Most of these accessories do not have floating features.

On the other hand, with the instant invention cranial-cervical and dorsum-lumbar immobilization is obtained in a single operation, thus decreasing the time required for immobilizing or moving the victim or patient, providing at the same time, heat, floating capacity, hemodynamic and cardiac monitoring.

### SUMMARY OF THE INVENTION

The object of the invention is allowing the intervening persons (physicians, policemen, firemen, rescue personnel, paramedicals, civil personnel) to carry out immobilization of the victim or patient in a quick and simple way, thus affording relief and safety to both, the patient and the person in charge of the task.

### BRIEF DESCRIPTION OF THE DRAWINGS

A preferred embodiment of the invention, which is merely an example in no way limiting the scope thereof, will be now described in connection with the accompanying drawings, in which:

FIG. 1 is a front view of the two half bodies comprising the device, showing its fixing and accessory components.

FIG. 1A is a front view similar to that of FIG. 1, in the long version thereof.

FIG. 1B is a scheme of the rear joint, slide, straight wedge, male and female fixing wedge, and upper and lower end fasteners or fixing means.

FIGS. 1C and D show fixing alternatives of rear joints.

FIG. 2 is a perspective view of the cranial-cervical fixing members.

FIG. 3 is a front and side view of the front portion of the cervical fixing means.

### DETAILED DESCRIPTION OF THE INVENTION

In all figures the same reference numerals designate the same or equivalent parts.

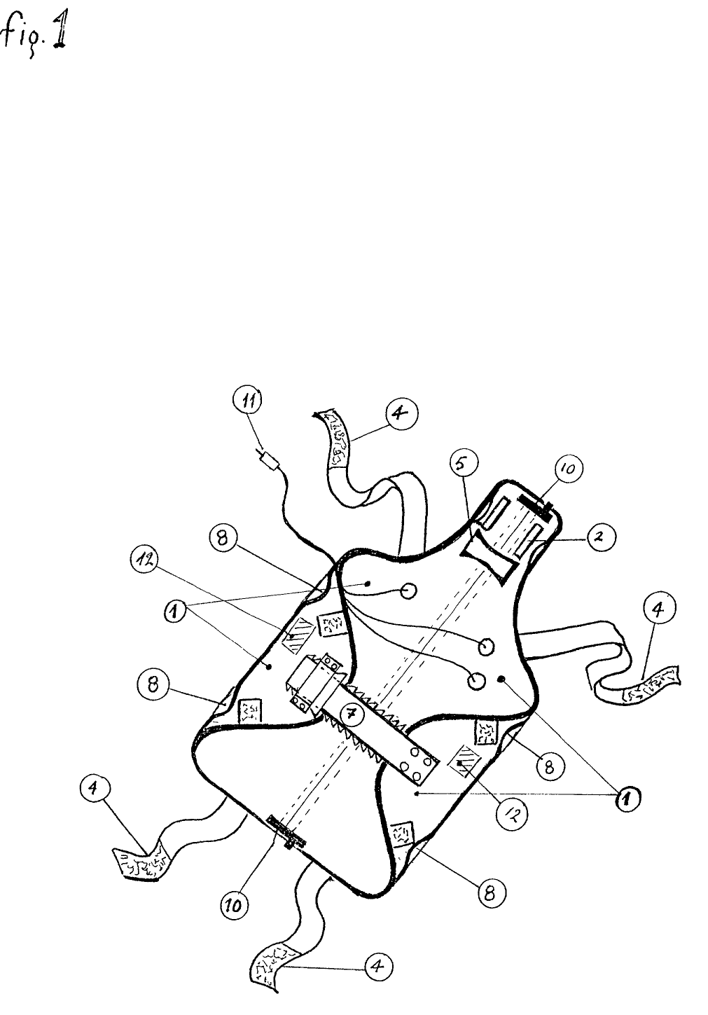

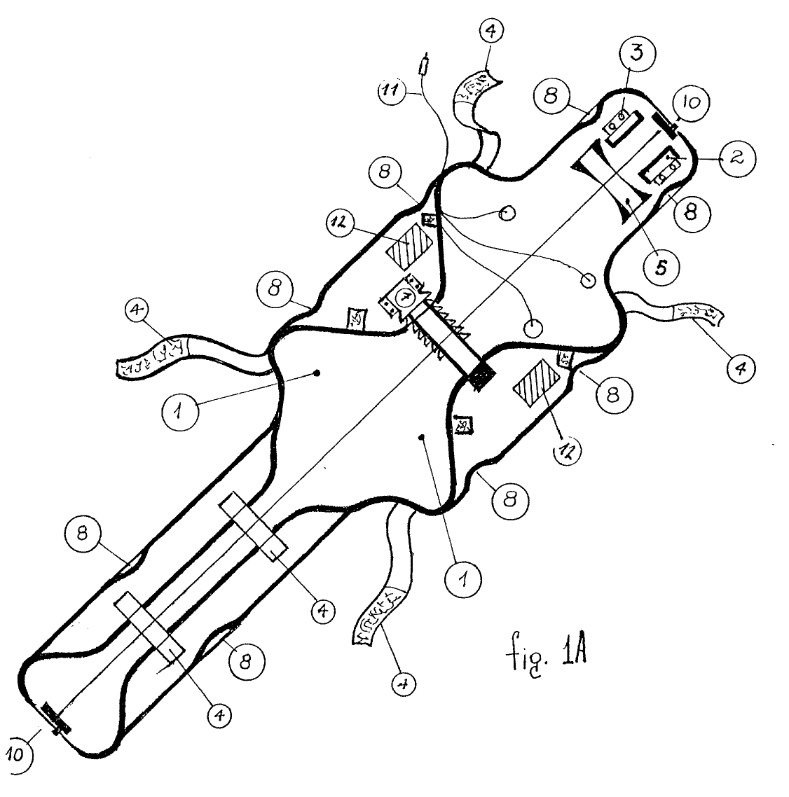

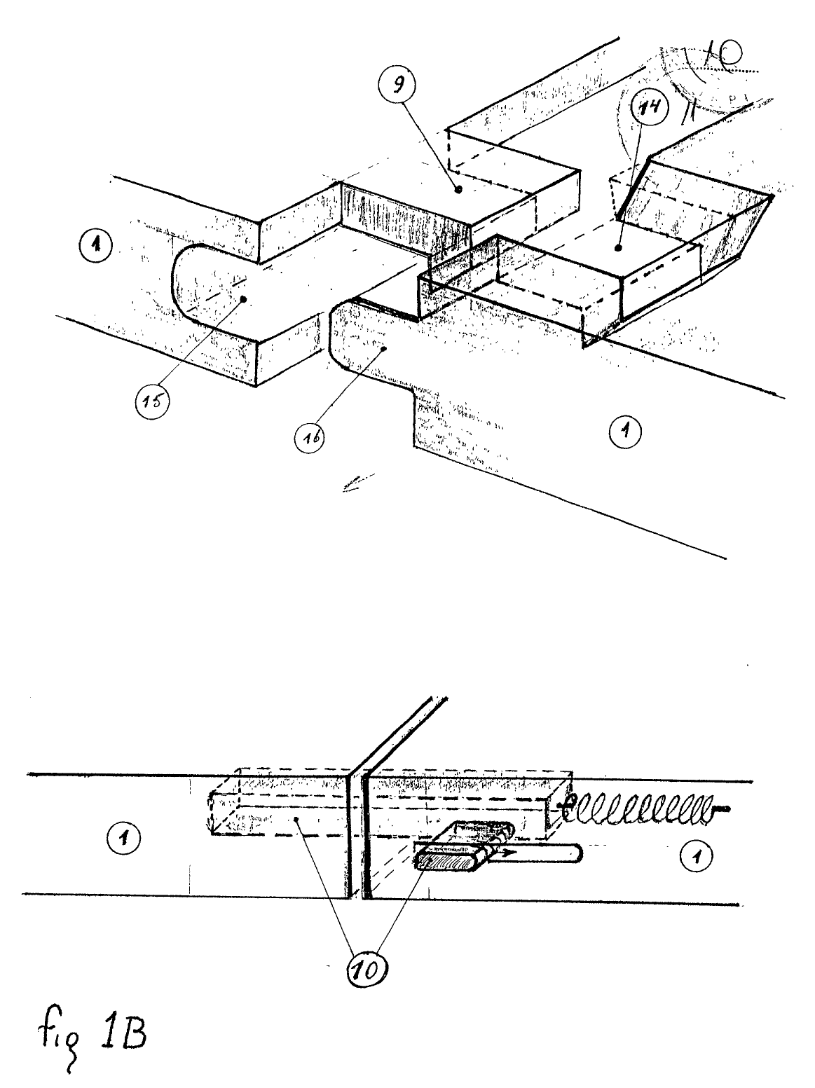

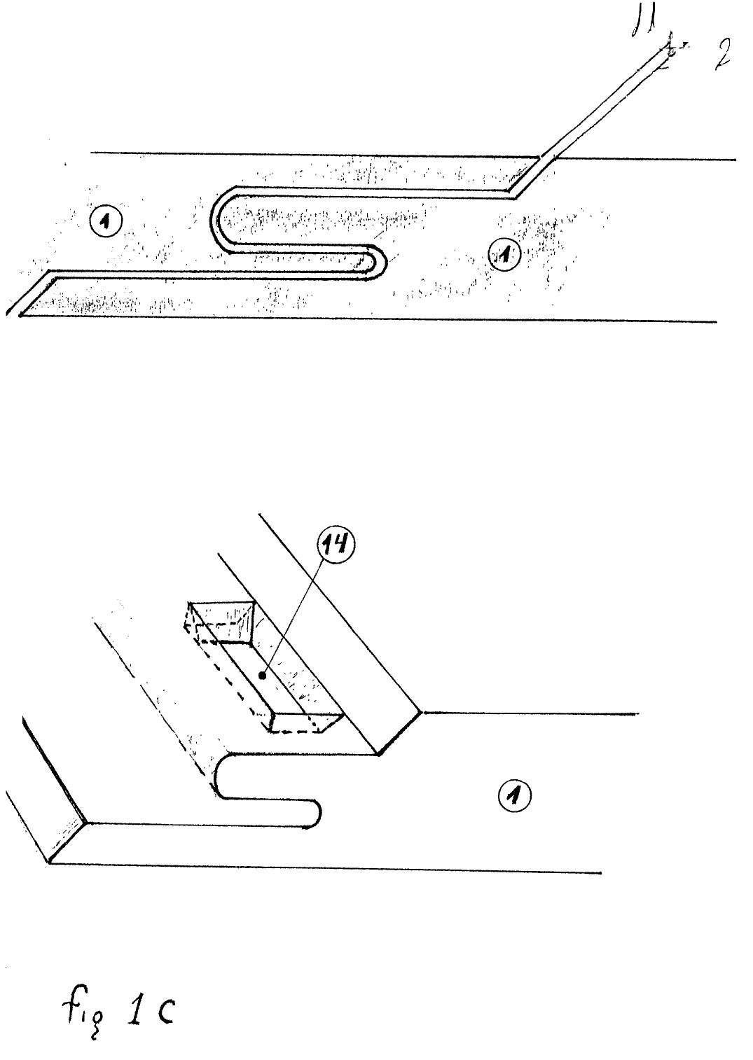

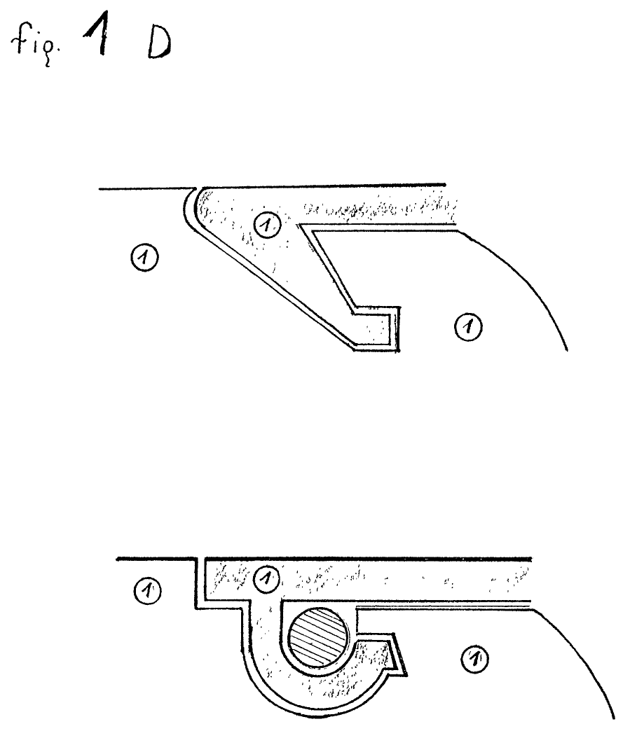

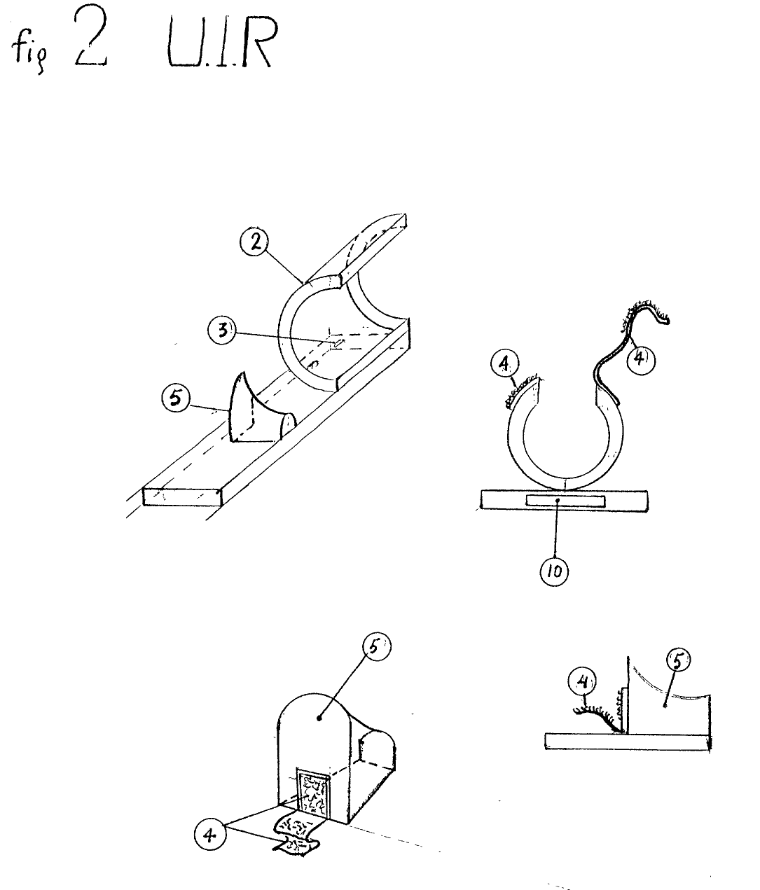

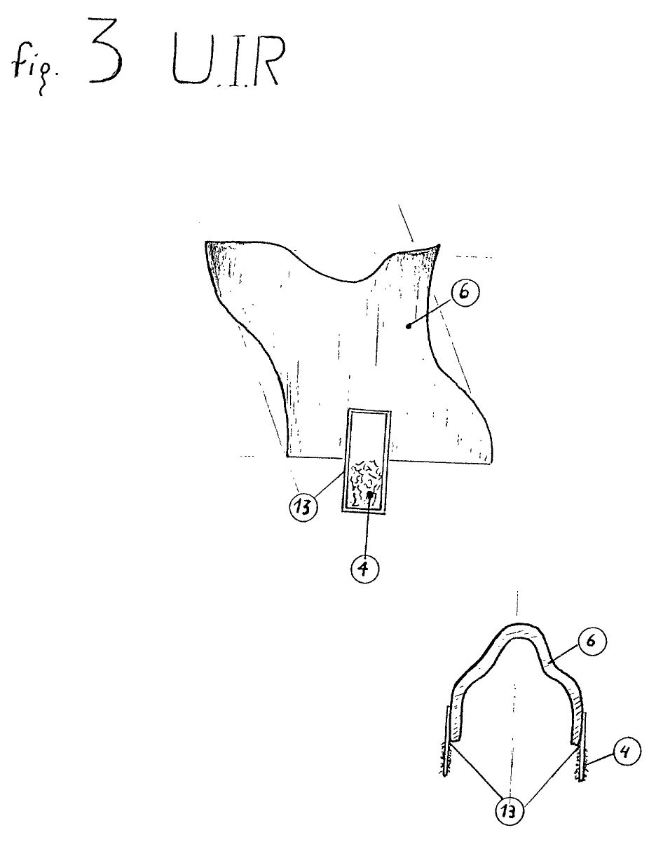

The quick immobilization unit of the invention is basically comprised by two parts made of translucent blown plastics, e.g. high impact PVC (1) which allows the inclusion of members and /or chemical substances affording floating capability, insulation and heat; which is constituted by two main symmetrical portions, which are simultaneously joined in their rear and front parts upon reaching the human body by both sides, right and left; each of the parts is a single piece folded over itself which has, along the joining (rear) sides, a channel along the left portion (15) mating with a straight wedge (16) at the right portion, as seen in FIG. 1B, the slide acting for joining both portions through wedges (9), which join into cavities (14) of the main bodies. Once stabilized, the device adopts an anatomic shape accommodating the cranial-cervical region, the thorax-abdominal region and the lower members. The long version shown in FIG. 1A leaves the upper thorax region of the patient free for RCP and monitoring actions; the rear portion once assembled protects the cranial, cervical and dorsum-lumbar regions in a single operation: at the upper and lower ends (i.e. the shoulders region) the device has fasteners of the Velcro® type (4) which are crossed with those of the front portion; at the center of upper and lower ends, the device has a pin lock (10), through a cavity in the main part, avoiding displacement of the main parts while such displacement is not required; a cranial fastening system (2), (adjustable to the cranial size) is comprised by two semicircles of solid plastics which are attached, each half to the corresponding main body, through bolts, plastic rivets (3) or high strength adhesive, with Velcro at their ends for completing fixation; it also has a cervical pillow (5) attached to the main body, with Velcro on the side faces acting as a joining element of the front part of the cervical immobilizing device (6); this immobilizing device has straps (13) at the side faces, covered by Velcro; on both sides the main parts have six cavities (8), or eight cavities in the long version, acting as safe handling means; the front part has at the four ends Velcro fasteners mating those already mentioned for the rear portion; at the front outer center it has a self-regulating track system (7), made of plastics, attached to the main body by bolts, plastic rivets or high strength adhesive.

Auxiliary elements such as a patient monitoring wiring (11), two pockets (12) for carrying the device measuring oxygen concentration and a cardiac monitor are included at the front part of the main body; and a medicine and emergency kit holder (17) (E.G. pre-filled syringes) at the upper part.

1. Quick Immobilization Device, made of translucent blown plastics, of the high impact PVC type, allowing the inclusion of elements and/or chemical substances affording floating capability, insulation and heat, for protecting victims or patients (adults or children) requiring immobilization for being moved or rescued, thus avoiding new lesions, progression of existing lesions or deterioration thereof, characterized by comprising two main symmetrical parts, which are simultaneously joined at their rear and front parts upon reaching the human body by both sides, right and left; each of the parts is a single piece curved over itself which has, along the joining (rear) sides, a channel along the left portion mating with a straight wedge at the right portion, the slide acting for joining both portions through wedges, which join into cavities of the main bodies; once stabilized, the device adopting an anatomic shape accommodating to the cranial-cervical region, the thorax-abdominal region leaving the upper thorax region of the patient free for RCP and monitoring actions; the rear portion once assembled protecting the cranial, cervical and dorsum-lumbar regions in a single operation; at the upper and lower ends (at the shoulders region) the device has fasteners of the Velcro® type which are crossed with those of the front portion; at the center of upper and lower ends, the device has a pin lock, through a cavity in the main part, avoiding displacement of the main parts when such displacement is not required; a cranial fastening system, (adjustable to the cranial size) comprised by two semicircles of solid plastics which are attached, each half to the corresponding main body, through bolts, plastic rivets or high strength adhesive, with Velcro at their ends for completing fixation; it also has a cervical pillow fixed to the main body, with Velcro at the side faces acting as a joining element of the front part of the cervical immobilizing device; this immobilizing device having straps at the side faces, covered by Velcro; on both sides the main parts have six cavities, or eight cavities in the long version, acting as safe handling means; the front part has at its four ends Velcro fasteners mating with those already mentioned for the rear portion; at the front outer center it has a self-regulating track system, made of plastics, attached to the main body by bolts, plastic rivets or high strength adhesive.

2. Quick immobilization device, as claimed in claim 1, characterized by allowing fixing and protection of patient's legs, comprised by two parts (extensions of former parts) made of plastics and folded over themselves, having two cavities allowing handling of the device, having at the upper part two Velcro straps completing the fixing action.

3. Quick immobilization device as claimed in claims 1 and 2, characterized by including at the front part accessories such as patient's monitoring wiring, two pockets for holding a device measuring oxygen concentration and a cardiac monitor, and at the upper part a medicine and emergency kit holder (e.g., syringes pre-filled with pharmacological preparations).
